(19) United States
(12) Patent Application Publication
(10) Pub. No.: US 2009/0196943 A1
(43) Pub. Date: Aug. 6, 2009

(54) Preventing/Treating signs of skin stress/aging by bioenergetically modifying acupuncture points of the face or neck

(75) Inventors: LIONEL BRETON, Versailles (FR); MICHEL FREY, Paris (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 12/320,672

(22) Filed: Jan. 30, 2009

Related U.S. Application Data
(63) Continuation of application No. 11/239,087, filed on Sep. 30, 2005, now abandoned.
Application No. 11/239,087 is a continuation of application No. PCT/EP04/04524, filed on Mar. 31, 2004.

(30) Foreign Application Priority Data
Apr. 1, 2003 (FR) 03/04032

(51) Int. Cl. A61K 8/97; A61Q 19/00; A61Q 19/08

(52) U.S. Cl. 424/736; 424/725; 424/764; 424/746; 424/745; 424/742; 424/747

(57) ABSTRACT

A regime or regimen for preventing and/or treating the signs of aging and/or stress of the skin, especially for (i) smoothing skin wrinkles and/or relaxing marks on the forehead and/or (ii) tonifying the cheeks, entails topically applying, advantageously via transdermal delivery, onto at least one acupuncture point of the face or the neck of an individual in need of such treatment, notably the acupuncture points PC-1, 6-E, 18-IG, 19-GI, 23-VC, or a restricted area of the face or the neck containing same, at least one bioactive agent modifying the bioenergetic environment at said at least one acupuncture application point and thus mimicking the effects of acupuncture, for example a relaxing/tonifying essential oil.

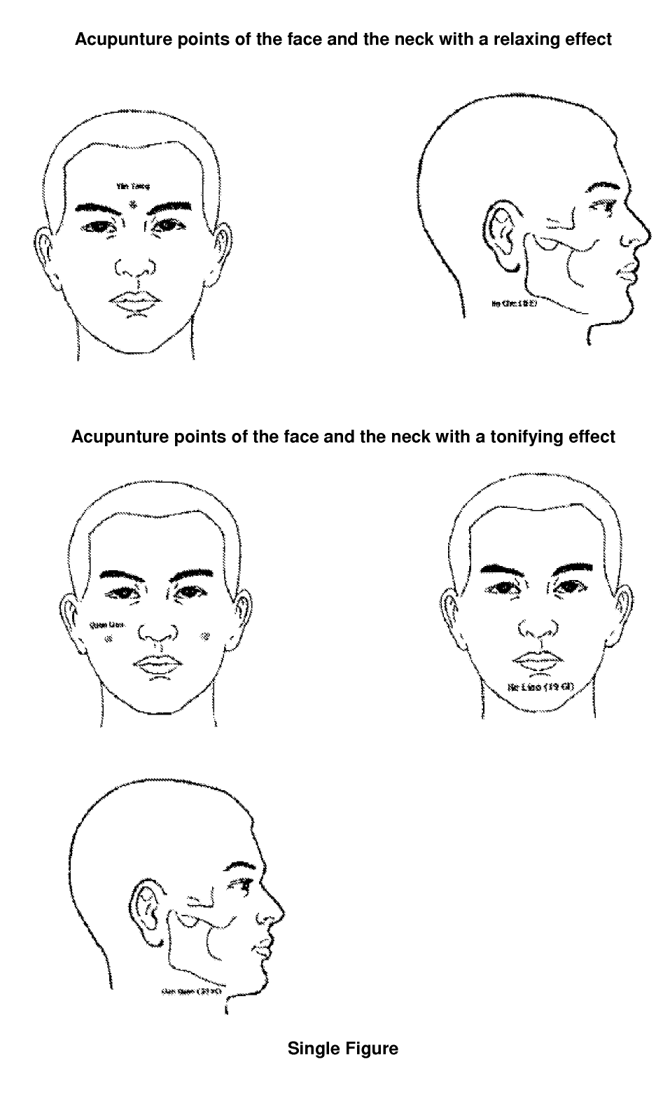

### CROSS-REFERENCE TO PRIORITY/PCT APPLICATIONS

This application claims priority under 35 U.S.C § 119 of FR 03/04032, filed Apr. 1, 2003, and is a continuation of PCT/EP 2004/004524, filed Mar. 31, 2004 and designating the United States (published in the English language on Oct. 14, 2004 as WO 2004/087090 A2), each hereby expressly incorporated by reference and each assigned to the assignee hereof.

### BACKGROUND OF THE INVENTION

1. Technical Field of the Invention

The present invention relates to a cosmetic process (regime or regimen) for preventing and/or treating the signs of aging and/or stress of the skin, comprising the topical application, to at least one acupuncture point of the face or the neck selected from among the points PC-1,6-E, 18-IG, 19-GI and 23-VC or a restricted area of the face and/or the neck comprising same, of a cosmetic system for topical application containing, in a physiologically acceptable medium, at least one substance/bioactive agent capable of modifying the bioenergetic environment at the said application point, the said system having a surface adapted to the individualized treatment of the said acupuncture points.

The expression “individualized treatment” means a localized treatment at each acupuncture point or on a restricted area of the face or the neck comprising a single acupuncture point.

The expression “cosmetic system for topical application” used in the process according to the invention means a cosmetic composition in a solid or semi-solid form, which is preferably self-adhesive, comprising at least one active substance according to the invention, which is capable, when the system is placed in contact with the skin, of modifying the bioenergetic environment at the said acupuncture point either by contact with the skin or by diffusion into the skin (transdermy). This will then be referred to as a “transdermal delivery system” or “transdermal system”, preferably employed in the process of the invention.

In particular, the system is in a form that allows a controlled release and/or diffusion of the said active substance, onto the skin or through the skin. Such a system advantageously comprises a reservoir layer comprising, for example, a gelled matrix or of a polymer matrix in which is incorporated a dose of active substance; it allows the controlled delivery of a unit dose of active substance. Preferred examples of such systems that may be mentioned include patches, hydrogels or composite films, advantageously cut beforehand into the form of a lozenge for localized application onto an acupuncture point according to the invention.

According to another embodiment, the system is in the form of unit doses of compositions (e.g., gel) which are capable of forming a film after application to the acupuncture point; the gel according to the invention will be applied to the acupuncture point with the tip of a finger or an applicator, so that the surface of the film thus formed on the skin is suitable for the individualized treatment of the acupuncture points, preferably from 1 to 5 cm².

The system used in the context of the invention differs from the standard cosmetic formulations described in the prior art (U.S. Pat. No. 5,925,348, WO 99/62481, U.S. Pat. Nos. 6,270,811 and 5,599,546) in that it is characterized by a unit (indivisible and single-dose) in solid or semi-solid form, which is preferably adhesive, having a surface that is suitable for localized application to an acupuncture point, which allows a release of a unit dose of active substance at the surface of or through the skin, locally at the said acupuncture point.

In particular, the substance may be an essential oil selected from an essential oil with a relaxing action and an essential oil with a tonifying action.

Women, and even men, currently have a tendency to wish to look youthful for as long as possible and consequently seek to fade out the signs of aging on the skin, which are reflected in particular by wrinkles and fine lines. In this respect, the media and the fashion world report about products intended to keep the skin radiant and wrinkle-free for as long as possible, which are signs of youthful skin, and all the more so since the physical appearance acts on the psyche and/or on the morale.

2. Description of Background and/or Related and/or Prior Art

Hitherto, wrinkles and fine lines were treated using cosmetic products containing active agents acting on the skin, for example by moisturizing it or by improving its cell renewal or alternatively by promoting the synthesis of collagen, which makes up skin tissue.

Although these treatments make it possible to act on the wrinkles and fine lines caused by chronological or intrinsic aging, and also on those caused by photo-aging, they have no effect on expression wrinkles, which require an intervention on the contractile muscle component of the wrinkles present in the skin. These expression wrinkles are in principle located radially around the mouth and/or the eyes and/or horizontally on the forehead and/or located in the space between the eyebrows.

Researchers of the assignee hereof have previously shown that the contractile muscle fibers, in particular striated muscle fibers, which are under the direct control of the neuromuscular impulse, play an essential role in the formation of expression wrinkles, and that modulating the neuromuscular impulse attenuates expression wrinkles and also has a “smoothing” effect on the skin's microrelief. Moreover, repeated mechanical tractions exerted by the subcutaneous muscles on the expression regions of the face (forehead and glabella wrinkles, and crow's feet) create tension forces in the dermis, which are thought to gradually modify the phenotype of the fibroblasts present in the area of the wrinkle, causing them to acquire particular contractile properties.

In parallel, the skin is subjected to a cutaneous stress, which may be of chemical, physical, mechanical or bacterial origin. Specifically, it is known that during a superficial cutaneous stress, the keratinocytes of the surface layers of the epidermis release biological mediators that have the capacity of attracting certain infiltrating cells of the skin, which are themselves responsible for maintaining a transient local irritation. These cells infiltrating the irritated or attacked areas then release enzymes such as leukocyte elastase, under the action of which, in particular, the elastic fibers providing extracellular support to the connective tissue may be degraded, and thus result in a reduction in the elasticity of the skin.

Thus, in the long term, the sum of the superficial cutaneous microstresses generated, for example, by a prolonged exposure to UV rays or by irritant agents, can result in a more or less accelerated loss of the natural elasticity of the skin. The network formed by the elastic fibers of the subjacent connective tissue and of the extracellular spaces can then gradually become destructured. This results in accelerated aging of the skin (wrinkled and/or less supple skin) via impairment of the dermal elastic network, and also an increase in wrinkles (deeper wrinkles). The main areas of slackening of the skin on the face and the neck are located on the cheekbones, the nasolabial fold and under the chin.

One of the means commonly used for acting on expression wrinkles has been botulinum toxin, which is in particular injected into the wrinkles of the glabella, which are the wrinkles between the eyebrows (see J. D. Carruters et al., J. Dermatol. Surg. Oncol., 1992, 18, pp. 17-21). Dermatologists also make use of degradable implants based on collagen, hyaluronic acid or polylactic acid. However, these methods have the drawback of requiring a medical intervention and do not give long-lasting results.

Cosmetic processes involving stimulation of the acupuncture points are also known, which use magnetic and/or electrical systems, or more traditionally acupuncture needles; however, these methods require the intervention of a practitioner and occasionally generate dysaesthetic sensations (heating or pain) for the patient.

Cosmetic patches with a calmative action (EP-0,976,382) or with an anti-wrinkle action (WO 03/063817, U.S. Pat. No. 5,785,978) are also known from the prior art, which are intended to be applied to the entire face or to extended areas of the face marked with wrinkles and/or stress, but these patches are not applied to particular acupuncture points, nor even are they adapted to the individualized treatment of acupuncture points. Furthermore, these patches applied to extended areas of the face must adhere well to the entire area to be treated, while at the same time remaining easy to remove without causing any irritation or redness after treatment, but must also have good elasticity for the comfort of the individual during the treatment, these all therefore being criteria that make them complex to formulate.

There is thus a need to find novel cosmetic processes for preventing and/or treating the signs of aging and/or stress of the skin, which are easier to implement and which do not have the drawbacks of the methods described above.

### SUMMARY OF THE INVENTION

It has now been determined, unexpectedly, that the application, to at least one acupuncture point of the face or the neck selected from among the points PC-1,6-E, 18-IG, 19-GI and 23-VC or a restricted area of the face or the neck comprising same, of a system for topical application, e.g., in the form of a patch or a hydrogel having an area of from 0.25 to 3 cm² and containing, e.g., at least one essential oil, makes it possible to modify the bioenergetic environment at the acupuncture point and thus to mimic the effects of acupuncture. This modification of the bioenergetic environment may entail:

a modification of the electrophysiological status of the cells by inducing a variation in the membrane potential of the skin cells, via the influx and efflux of cations or anions to modify their excitation threshold;

and/or a modification of the electromagnetic environment of the skin cells;

and/or an action on the subcutaneous capillary circulation, for example by vasodilation or vasoconstriction;

and lead, depending on the application points, to a relaxed and/or destressed skin (points PC-1 and 6-E) or, on the contrary, to a tonified and/or firmed skin (points 18-IG, 19-GI and 23-VC), and to do so in a manner extended to the area marked by signs of aging and/or stress of the skin, comprising the said acupuncture point, via a “remote” effect mediated by stimulation of the acupuncture point.

Acupuncture is based on an energy model in which the energy circulates along specific pathways known as meridians, each meridian being associated with a particular physiological system and a specific internal organ. The acupuncture points are specific sites along the meridians, each point having a predictable effect on the vital energy.

This process combining (i) transdermal systems with a surface adapted to the individualized treatment of the said acupuncture points and comprising at least one substance capable of modifying the bioenergetic environment at the application point and (ii) acupuncture points of the face or the neck with a relaxing effect (points PC-1 and 6-E) or with a tonifying effect (points 18-IG, 19-GI and 23-VC) has the advantage of being easy to implement and of not giving rise to any dysaesthetic sensations for the patient; on the contrary, the patient even has a pleasant impression of relaxed and/or firmed and visibly rejuvenated skin.

The use of such systems with a surface adapted to the individual treatment of the said acupuncture points also makes it possible, compared with the cosmetic patches previously described for application to the entire face or to extended areas of the face or the neck:

to provide a system that is easier to formulate (fewer constraints as regards the criteria of adhesion, elasticity and comfort as a result of their small size) and that allows the metering of active substance to be applied (e.g., one unit dose per system); also easier to apply and to remove, with good adhesion to the skin on the said acupuncture points, and which is very comfortable during the time of application, due to its small size;

to treat, via a “remote” effect mediated by the stimulation of the acupuncture point on a skin surface of a few mm², an extended area of wrinkles or of slackening of the skin that may be up to several cm² around the said acupuncture point;

to individually treat restricted areas of the face or the neck, as a function of the needs of the individual, by application of specific systems (relaxing or tonifying systems) selectively on the corresponding acupuncture points;

to adapt the formulation (choice of active agent) of the said systems as a function of the acupuncture point to be treated: relaxing agents may thus preferably be used in systems intended for the relaxing points PC1 and 6E and tonifying active agents may preferably be used in systems intended for the tonifying points 18IG, 19GI and 23C;

to adapt the dose of the active agent as a function of the system (size, mode of diffusion: passive, active, controlled release, etc.) and of the acupuncture point to be treated.

### DETAILED DESCRIPTION OF BEST MODE AND SPECIFIC/PREFERRED EMBODIMENTS OF THE INVENTION

The present invention thus features a cosmetic regime or regimen for preventing and/or treating the signs of aging and/or stress of the skin, comprising the application, to at least one acupuncture point of the face or the neck selected from the points PC-1, 6-E, 18-IG, 19-GI and 23-VC or a restricted area of the face or the neck comprising same, of a system for topical application containing, in a physiologically acceptable medium, at least one substance capable of modifying the bioenergetic environment at the said application point, the said system having a surface adapted to an individualized treatment of the said acupuncture points.

The substance capable of modifying the bioenergetic environment at the said application point will advantageously be selected from the group consisting of trace elements, precious metal salts, precious stones dusts or semi-precious stones dusts, vasoconstrictors, vasodilators and essential oils, and a mixture thereof.

According to the invention, the “signs of aging of the skin” are especially the formation of wrinkles and fine lines, in particular expression wrinkles.

According to the invention, the “signs of stress of the skin” are especially impairment in the dermal elastic network leading to a loss of elasticity and slackening of the skin.

According to the invention, the “acupuncture points of the face or the neck” are characterized in that they are:

(i) localized at the areas of formation of wrinkles (forehead, glabella, crow's feet) and/or at the areas of tension (front of the mandibular gonion) and associated with a relaxing energy; or

(ii) localized at the areas of relaxation of the skin (under the chin, nasolabial fold, cheekbones), and associated with a tonifying energy.

The acupuncture points with a “relaxing” or “tonifying” effect of the face and/or the neck described in the Dictionnaire des points d'acupuncture [Dictionary of acupuncture points] by Dr. G. Guillaume and Dr. M. Chieu will especially be used.

The points with a “relaxing” effect are:

PC1-Yin Tang, located on the forehead (area of formation of wrinkles), between the two eyebrows in the glabella; it has a relaxing activity;

6E-Jia Che, located at the front of the mandibular gonion (area of tension); it releases tension from the masseters in the case of contracture;

The points with a “tonifying” effect are:

18IG-Quan Liao, located directly underneath the outer canthus, in a depression located on the lower edge of the zygomatic arch; it disperses wind, invigorates the Luo (tonifies the energy in the meridian transfers), permeabilizes the meridians and calms pain;

19GI-He Liao, located on either side of the nasolabial fold; it tonifies the sagging of the nasolabial fold;

23VC-Lian Quan, located under the chin; it reinforces the elasticity of the epidermis in the area under the chin.

The location of these points is shown in the attached FIGURE of Drawing.

The expression “restricted area of the face or the neck comprising the said acupuncture point” means an area comprising one of the acupuncture points according to the invention taken individually, the said area not extending to the entire face or neck. For example, this area may correspond to the size of the system applied to the said acupuncture point, generally of from 0.25 cm² to 10 cm², preferably from 0.25 to 5 cm² and even more preferably from 0.25 to 1 cm².

Preferably, the cosmetic system for topical application is a transdermal delivery system or “transdermal system”, i.e., a system allowing diffusion of the substance capable of modifying the bioenergetic environment at the said acupuncture point through the skin (transdermy).

Advantageously, the cosmetic system for topical application according to the invention comprises a reservoir layer in which is incorporated the active substance, allowing a controlled release of the said active substance. Such a reservoir layer may be a gelled matrix or a polymer matrix of hydrophilic or hydrophobic type in which the said substance is incorporated.

The said system preferably has a surface suitable for individualized application to an acupuncture point of from 0.25 to 10 cm², preferably from 0.25 to 5 cm² and even more preferably from 0.25 to 1 cm². For example, the system has an area of 0.25, 0.5, 1, 2 or 3 cm².

A preferred system for use in the process of the invention is a system in the form of a patch, a hydrogel or a composite film, preferably a patch or a hydrogel, cut beforehand into the shape of lozenges of small area that are suitable for an individualized treatment of the acupuncture points.

In particular, the substance capable of modifying the bioenergetic environment at the acupuncture point may be selected from among agents acting on the electrophysiological status of the skin cells, by inducing a variation in the membrane potential of the skin cells, especially by influx and efflux of cations or anions to modify their excitation threshold; agents modifying the electromagnetic environment of the skin cells around the acupuncture point; agents acting on the cutaneous capillary circulation, for example by vasodilation or vasoconstriction; and mixtures thereof.

This substance may be of natural or synthetic origin.

The term “natural origin” means a compound in pure form or dissolved at various concentrations, obtained by various extraction processes, especially from a plant extract of natural origin.

The term “synthetic origin” means a compound in pure form or dissolved at various concentrations, obtained chemically or by production in an organism after introduction into this organism of the elements required for this production.

As examples of “agents acting on the electrophysiological status of cells” at the application point, mention may be made of trace elements, for example copper, zinc, selenium, germanium, iron, calcium, magnesium and manganese, and mixtures thereof. Examples that may be mentioned include the gluconate, citrate or carbonate of the above metals. Zinc may advantageously be used, zinc being a mineral that is essential for the correct functioning of the body as an indispensable stimulant of the immune system, combined with selenium, which is a powerful antioxidant and detoxifying agent.

These compounds generally represent an amount of from 0.1% to 10% by weight and preferably from 1% to 5% in total.

As examples of “agents modifying the electromagnetic environment around the acupuncture point”, mention may be made of noble metal salts, as gold salts, silver salts, platinum salts, titanium salts, or precious stones dusts or semi-precious stones dusts, as diamond dusts, ruby dusts, emerald dusts or sapphire dusts for example.

Examples of “agents acting on the capillary circulation” (vasoprotector or vasodilator) that may be selected include flavonoids, ruscogenins, esculosides, escin extracted from common horse chestnut, nicotinates, hesperidine methyl chalcone, essential oil of lavender and astringent plant extracts or stimulating plant extracts.

Astringent plants are known for their roughness in mouth or their metallic taste; mention may be made of extracts of chestnut, artichoke, may, or tannic extracts.

As examples of stimulating plants, mention may be made of ginseng, rhodiola rosea-russa, coriander, saffron or peppermint.

The amount of these agents may vary within a wide range. In general, these active agents are present in a concentration ranging from 0.01% to 15% and preferably from 0.05% to 10% by weight relative to the total weight of the composition.

According to one preferred embodiment, an essential oil will be used as substance capable of modifying the bioenergetic environment at the acupuncture point.

The expression “essential oil” according to the invention means a volatile oil naturally produced by secretory organs (flower, leaf, root, seed) of several plants. These volatile oils comprise terpenes (carbon hydrates non aromatic for 95%) and oxygenated compounds (alcohols, aldehydes, ketones) which are aromatic.

These essential oils are extracted by physical means, direct cold expression or driven with water vapor.

Natural or synthetic essential oils may be used, for instance eucalyptus oil, dill oil, lavandin oil, lavender oil, marjoram oil, vetiver oil, litsea cubeba oil, lemon oil, sandalwood oil, camomile oil, savory oil, clary sage oil, nutmeg oil, cinnamon oil, hyssop oil, caraway oil, orange oil, peppermint oil, grapefruit oil, geraniol oil, cade oil, bergamot oil, thyme oil, mace oil, tea tree oil, ylang-ylang oil, rose pellargonium oil, rosewood oil, rock rose oil, lemongrass oil, petitgrain oil and ravensare oil, and mixtures thereof.

The following are especially distinguished:

essential oils with a “relaxing” action, for instance essential oil of lavender, of ylang-ylang, of camomile, of sandalwood, of clary sage, of thyme, of marjoram, of grapefruit or of jasmine, or a mixture thereof. An essential oil of lavender, of marjoram or a mixture thereof, which are known for their antispasmodic, sedative and bactericidal action, will preferably be used;

essential oils with a “tonifying” action, for instance essential oil of eucalyptus, of vetiver, of lemon, of lemongrass, of litsea cubeba, of peppermint, of orange, of petitgrain, of ravensare, of bergamot, of tea tree and of mace or a mixture thereof. Essential oil of tea tree, of mace or a mixture thereof will preferably be used.

The essential oil in the composition according to the invention is preferably present in an amount of from 0.01% to 20% of the total weight of the composition, preferentially from 0.1% to 10% of the total weight of the composition and even more preferentially from 0.5% to 5% of the total weight of the composition.

According to a first embodiment, the process according to the invention is directed towards preventing and/or smoothing out wrinkles and fine lines, in particular expression wrinkles, and/or relaxing the lines of the face; it comprises the application to at least one acupuncture point of the face or the neck selected from the relaxing points PC-1 and 6-E or to a restricted area of the face comprising it, of a transdermal system containing, in a physiologically acceptable medium, at least one essential oil selected from the essential oils of lavender, of marjoram, of ylang-ylang, of tea tree and of mace, and a mixture thereof.

Alternatively, an essential oil with a relaxing action or an essential oil with a tonifying action may be used, either one being capable of inducing bioenergetic movements at the said acupuncture points devoted to relaxation.

For a potentiated relaxing effect, an oil with a relaxing action as defined above will preferably be used, preferentially selected from among lavender oil, marjoram oil and/or ylang-ylang oil.

According to a second embodiment, the process according to the invention is directed towards firming and/or tonifying the skin; it comprises the application to at least one acupuncture point of the face or the neck selected from among the tonifying points 18-IG, 19-GI and 23-VC or to a restricted area of the face or the neck comprising it, of a transdermal system containing, in a physiologically acceptable medium, at least one essential oil selected from among the essential oils of lavender, of marjoram, of ylang-ylang, of tea tree and of mace, and mixtures thereof.

Alternatively, an essential oil with a relaxing action or an essential oil with a tonifying action may be used, either one being capable of inducing bioenergetic movements at the said acupuncture points devoted to stimulation.

Preferentially, an essential oil with a tonifying action as defined above will be used, preferably selected from the essential oils of mace and of tea tree, to obtain a potentiated tonifying effect at the application points.

A preferred process according to the invention is directed simultaneously towards relaxing the forehead and firming the cheekbones of the face; it comprises the application of a transdermal system containing at least one essential oil with a relaxing action to the relaxing point PC1 or to a restricted area of the face comprising it and of a transdermal system containing at least one essential oil with a tonifying action to the tonifying point 18-IG or to a restricted area of the face comprising it.

A lavender oil, ylang-ylang oil or marjoram oil will preferably be used on the point PC1 or on a restricted area of the face comprising it and an essential oil of mace or of tea tree will preferably be used on the point 18-IG or on a restricted area of the face comprising it. The application will be performed simultaneously on the two points 18-IG of the cheekbones.

The composition of the transdermal system in the process according to the invention may also comprise at least one compound selected from among plant extracts and vitamins, or mixtures thereof.

Preferred plant extracts according to the invention that may be mentioned include:

Cang Zhu, which is a Chinese plant whose root is used in the form of a tincture (10%); its main function is to modulate the opening of the ion channels;

green tea, the leaves of which are used in the form of an aqueous-alcoholic tincture (10%); the leaf contains polyphenols with antioxidant and anti-inflammatory properties allowing a reduction in the production of oxidizing agents in the skin;

acerola, which contains a large amount of vitamin C; this plays a major role in the body's defense processes; its antioxidant action combined with selenium prevents and combats aging;

Ginkgo biloba, rich in flavonoids, which promotes tissue regeneration, regulates vascular stasis in the capillaries and inhibits peroxidation of the membrane lipids.

As vitamins that may be used according to the invention, mention may be made of the following vitamins: A, D2, B1, B2, B5, B6, B12, C and E, and also nicotinic acid (vitamin PP) and biotin (vitamin H), and mixtures thereof. For example, the combination vitamin B5+B12 will advantageously be used: these vitamins safeguard the survival of cells and promote their growth; in particular, the vitamin B5/vitamin B12 couple increases the resistance to stress and fatigue.

The plant extracts and vitamins are generally in amounts of from 0.01% to 10% and preferably from 0.5% to and 5%.

The process according to the invention may administer a composition in the galenical forms conventionally used for topical application and especially in the form of dispersions of the lotion or serum type, emulsions of liquid or semi-liquid consistency of the milk type, obtained by dispersing a fatty phase in an aqueous phase (O/W) or conversely (W/O), or suspensions or emulsions of soft, semi-solid or solid consistency of the cream or gel type, or alternatively multiple emulsions (W/O/W or O/W/O emulsions), microemulsions, vesicular dispersions of ionic and/or nonionic type, or wax/aqueous phase dispersions, the said compositions being packaged in single-dose form. Advantageously, single doses of polymer-based compositions (e.g., gel) capable of forming a film on the skin with a surface adapted for an individualized treatment of the acupuncture points will be used. These compositions are prepared according to the usual methods.

When the composition is in the form of an emulsion, the proportion of the oily phase of the emulsion may range, for example, from 5% to 80% by weight and preferably from 5% to 50% by weight relative to the total weight of the composition. The oils, emulsifiers and co-emulsifiers used in the composition in emulsion form are selected from among those conventionally used in cosmetics or dermatology. The emulsifier and co-emulsifier are generally present in the composition in a proportion ranging from 0.3% to 30% by weight and preferably from 0.5% to 20% by weight relative to the total weight of the composition. The emulsion may also contain lipid vesicles.

Moisturizers may also be used, in particular the 2-(methacryloyloxyethyl)phosphorylcholine/butyl methacrylate copolymer (90/10) as a 5% solution marketed by Nippon Oil and Fats.

Examples of oils that will be used include hydrocarbon-based oils of plant origin, synthetic esters and synthetic ethers, and silicone oils, and mixtures thereof.

Surfactants may also be added to homogenize the mixture of the aqueous and oily phases.

Advantageously, the compositions that will be used are compositions based on polymers with adhesive properties capable of forming a film that is removed after drying (EP-0,514,760, EP-0,826,364) or an elastic and flexible solid layer removed by applying a tissue or an adhesive plastic (WO 93/05893); or gels or pastes (“beauty masks”) which, after application to a localized area of the face, dry to give a film that is removed by washing, cleansing or peeling; this will generally be referred to as a “varnish patch” or a “peel patch”.

However, transdermal systems in a solid or semi-solid indivisible form will preferably be used, such as:

patches, i.e., any system for delivering an active agent, which has a composite structure in the form of layers which, after application to the skin, releases the active product via transdermy. Examples that may be mentioned include:

controlled-release patches with a hydrophobic polymer matrix (FR-2,738,744 L'Oreal);

patches of reservoir type containing a reservoir of active substance, a diffusion membrane and an adhesive layer;

patches with optimized adhesion, comprising a layer of hydrophobic polymer attached to a support layer and containing particles of active compound, oil particles and particles of a water-absorbing agent (FR-2,761,889 L'Oreal);

gel patches with a high water content, of hydrogel type, especially based on at least one hydrocolloid, and having a contact adhesion similar to that resulting from a ventouse effect; these hydrogels have a fresh sensation when applied and give a great impression of softness;

oily gel patches based on copolymers of styrene and of isoprene, which may contain a large proportion of hydrophobic compounds;

composite films as described in EP-A-0,285,563; a composite film comprising a reservoir layer consisting of a matrix formed from a silicone polymer inside which are inclusions consisting of the active aqueous phase gelled by means of at least one gelling agent, will advantageously be used (FR-2,650,747 L'Oreal);

solid thin films based on natural or synthetic polymers, which, when applied to premoistened skin, dissolve in situ (WO 02/053197);

and advantageously “reservoir” systems allowing a controlled release of the said active substance.

Patches or hydrogels will preferably be used. Hydrogels containing an essential oil with a relaxing or tonifying action are the preferred formulations according to the invention, since they provide, in addition to the relaxing or tonifying effect, a very pleasant fresh effect when applied, and an impression of great softness.

The varnish patch may comprise water, polyvinyl alcohol, at least one vinylpyrrolidone copolymer and at least one oil, the polyvinyl alcohol being present in an amount ranging from 5% to 25% by weight relative to the total weight of the composition, as described in EP-1,186,291 (L'Oreal). The varnish patch is in the form of a gel to be spread over a limited area and may constitute a mask that has the advantage of being removed easily in one go, after a drying time of about 10 to 20 minutes.

The solid thin film may comprise natural or synthetic polymers as described in patent application WO 02/053197.

The patch generally comprises at least one polymer matrix, at least one surface of which, which is adhesive or capable of becoming so, especially after hydration, is intended to be placed in contact with the skin.

The term “polymer matrix” means a layer of hydrophobic or hydrophilic polymer that may consist of a self-adhesive matrix (on dry skin and/or on moistened skin).

The constituent “hydrophobic” polymer matrices of “standard” patches are especially based on polyacrylic or polyvinyl adhesive or a silicone, polyurethane, styrene or isoprene polymer whose crosslinking is preferably partial so as to give it adhesion without requiring an additional adhesive layer. A latex or butyl adhesive matrix or any other elastomeric adhesive may also be used.

The constituent “hydrophilic” polymer matrices of the “hydrogel” patches are especially based on natural or modified polysaccharides, polyacrylates, acrylamide, pyrrolidone or caprolactam derivatives and copolymers thereof, or proteins. They generally comprise at least one hydrocolloid that may be selected, for example, from the group consisting of: gellan gum; cellulose or its derivatives; algal extracts such as agar-agar, carrageenans and alginates; seed extracts such as carob gum, guar gum, guar gums modified especially by grafting an alkyl group; plant or microorganism exudates; fruit extracts such as pectins; gelling agents of animal origin such as gelatin or caseinates; water-soluble gelling synthetic polymers such as polyacrylic acids, optionally crosslinked with an alkyl chain, such as the “Carbopol” or “Pemulen” products from the company Goodrich; silicon derivatives such as synthetic hectorites, for instance the products “Laponite RD and RDS” marketed by Waverly, aluminum magnesium silicates, for instance the product “Veegum” marketed by Vanderbilt; products of the Polycaree type marketed by Rhone-Poulenc; or a mixture of these compounds.

They generally contain a high content of water, which may range, for example, from 30% to 90% by weight relative to the total weight of the hydrogel; this high content of water makes it possible to capture the skin's heat and to induce a sensation of freshness, resulting from the lowering of the temperature of the hydrogels that arises during the evaporation of the water contained in the hydrogel.

Advantageously, the polymer matrix may contain particles of at least one water-absorbing agent uniformly dispersed in the said matrix. Specifically, on contact especially with moisture from the skin, the particles of the water-absorbing agent take up water, thus promoting the dissolution of the water-soluble solid active compound. By means of this “in situ” solubilization of the water-soluble active agent, its bioavailability is, as it were, virtually instantaneous, and any possible interaction with the other compounds present in the polymer layer is minimized. The moisture of the skin can act as the solubilizing agent for the water-soluble active agent, and can do so all the more since the support layer of the patch and/or the matrix can create occlusive conditions.

Among the water-absorbing agents that may be present in the hydrophobic polymer matrix in dispersed form, mention may preferably be made of superabsorbent crosslinked polyacrylates with a high degree of swelling in water, such as those marketed by NoRsoLoR under the name Aquaplane®; polyvinyl alcohol; carboxyvinyl polymers such as those marketed by Goodrich under the name Carbopol®; semi-synthetic cellulose derivatives such as carboxymethylcellulose; natural substances such as starches, natural gums (guar gum, gum arabic or gum tragacanth), casein, phytocolloids (carrageenates, alginates or agar-agar), cotton fibers and gelatin.

It is also possible to use superabsorbent crosslinked polyacrylates, whose presence in dispersed form in a hydrophobic polymer matrix makes it possible, for example, to pump perspiration, and promotes, after hydration, better contact with the particles of the other active agents that may be present in the matrix.

The water-absorbing agent as defined above is preferably present in a proportion ranging from about 0.2% to about 20% by weight and more particularly ranging from 0.5% to 10% relative to the total weight of the polymer layer.

Besides the polymer(s) and water, the hydrogels may also contain a solvent selected from primary alcohols such as ethanol and isopropanol or one or more polyols, which are compounds comprising at least two hydroxyl functions. Examples of polyols that may be mentioned include glycerol, propylene glycol, butylene glycol, hexylene glycol, polyethylene glycols and dipropylene glycol, and mixtures thereof. The amount of polyol(s) may range, for example, from 10% to 40% by weight and preferably from 15% to 30% by weight relative to the total weight of the hydrogel.

The surface of the matrix intended to come into contact with the skin may be smooth or rough or may comprise relief.

Preferably, the polymer matrix is placed on a support. Such a support may consist of any suitable material that is impermeable to the active compounds contained in the adjacent matrix.

The support may be an “occlusive” support. By way of example, the support consists of a thermoplastic material selected from among high-density and low-density polyethylenes, polypropylenes, polyvinyl chlorides, copolymers of ethylene and of vinyl acetate, polyesters and polyurethanes, or a complex of such materials. These materials may also be present in laminated form with at least one sheet of metal such as a sheet of aluminum.

The support layer may be of any suitable thickness that will afford the desired support and protective functions. Preferably, the support layer is from about 20 μm to about 1.5 mm in thickness. Advantageously, the support layer is sufficiently flexible so as to be able to perfectly marry the profile of the skin, and so as not to cause the user any sensation of discomfort.

Preferably, however, the support is “non-occlusive”. In this latter case, a support consisting of a paper, a porous or perforated thermoplastic material, a woven, a nonwoven or a perforated nonwoven is advantageously used.

Preferably, the patch comprises at least one protective sheet, which can be peeled off before applying the patch.

In a known manner, the patches or hydrogels according to the present invention may be cut along a suitable contour corresponding to the area of skin surface to be treated defined by the acupuncture point to be treated. In general, the size or area of a patch in accordance with the invention is from 0.25 cm² to 10 cm², preferably from 0.25 to 5 cm² and even more preferably from 0.25 to 1 cm².

The patches or hydrogels according to the invention may advantageously be in a circular form that is particularly suitable for application to an acupuncture point; they may be in the form of a lozenge having an area of from 0.25 to 3 cm², for example 0.25, 0.5, 0.75, 1, 1.5, 2, 2.5 or 3 cm².

The patches or hydrogels may moreover be identified by a different color code, depending on whether it is a patch or hydrogel with a relaxing effect (for example green) or a patch or hydrogel with a tonifying effect (for example yellow). This color code may be incorporated directly into the polymer matrix by incorporation of colored pigments, or preferably onto the surface that is not in contact with the skin, or into the system for packaging said patches or hydrogels.

The patch or hydrogel may be packaged in a protective tray or sachet formed from two sheets of a leaktight paper/plastic film complex, the paper being coated with a room-temperature-weldable adhesive, the sheets being welded around the patch by contact of the faces coated with adhesive.

Preferably, the transdermal system according to the invention is used at the acupuncture point for an application time of between 1 minute and 120 minutes and preferably between 15 minutes and 30 minutes; however, the application time may be longer, for example overnight.

A cosmetic treatment kit comprising (1) at least one transdermal system containing at least one essential oil with a relaxing or tonifying action and (2) instructions for use indicating the location of the acupuncture points of the face and/or the neck, respectively in (i) the areas of formation of wrinkles and/or of tension or (ii) the areas of slackening of the skin.

This kit may comprise green hydrogels corresponding to a formulation based on an essential oil of lavender or of marjoram as described in Example 4, and yellow hydrogels corresponding to a formulation based on an essential oil of mace or of tea tree as described in Example 5.

The instructions for use will indicate the location of the relaxing or tonifying acupuncture points onto which the said hydrogels are to be applied, as described in the attached FIGURE.

This invention also features the cosmetic administration, on at least one acupuncture point of the face and/or the neck selected from the points PC-1, 6-E, 18-IG, 19-GI and 23-VC, of a transdermal system containing, in a physiologically acceptable medium, at least one substance capable of modifying the bioenergetic environment as defined above at an acupuncture point, as a substitute for implanted and/or electrical acupuncture systems, to prevent and/or treat the signs of aging and/or stress of the skin.

The appended FIGURE of Drawing shows the location of the acupuncture points of the face and/or the neck that are specific for the “relaxing” or “tonifying” effect, for application of the compositions or transdermal systems as defined according to the invention.

In order to further illustrate the present invention and the advantages thereof, the following specific examples are given, it being understood that same are intended only as illustrative and in nowise limitative In said examples to follow, all parts and percentages are given by weight, unless otherwise indicated.

### EXAMPLES

### Example 1

### Preparation of a “Relaxing” Composition

Formulation Based on Essential Oil of Lavender:

Oily Phase:

| Liquid petroleum jelly | qs 100% |
|---|---|
| Essential oil of lavender | 1% |

Aqueous phase

| | |
|---|---|
| 2-(Methacryloyloxyethyl)phosphoryl- | 3% |
| choline copolymer (Polyquaternium-51) | |
| Water | 3% |

Formulation Based on Essential Oil of Marjoram:

Oily Phase:

| Liquid petroleum jelly | qs 100% |
|---|---|
| Essential oil of marjoram | 1% |

Aqueous phase

| | |
|---|---|
| 2-(Methacryloyloxyethyl)phosphoryl- | 3% |
| choline copolymer (Polyquaternium-51) | |
| Water | 3% |

The two compositions are prepared according to the standard methods: the compounds of the oily phase are mixed together at 60° C. and the temperature is lowered to 40° C. In parallel, the compounds of the aqueous phase are mixed together at 80° C. The aqueous phase is then dispersed in the oily phase. The formulations are then packaged in drop-dose containers.

A drop will thus be easily applied to the defined acupuncture point.

### Example 2

### Preparation of a “Relaxing” Varnish Patch

Oil-in-Water Formulation Based on Essential Oil of Lavender:

| | |
|---|---|
| Essential oil of lavender | 0.1% |
| Sorbitol | 1.75% |
| Xanthan gum | 0.30% |
| Polyvinyl alcohol | 8.00% |
| Denatured alcohol | 20.00% |
| Water | 64.35% |
| Glycerol | 2.00% |
| PEG-75 | 2.00% |
| PEG-60 hydrogenated castor oil | 0.60% |

Oil-in-Water Formulation Based on Essential Oil of Marjoram:

| | |
|---|---|
| Essential oil of marjoram | 0.1% |
| Sorbitol | 1.75% |
| Xanthan gum | 0.30% |
| Polyvinyl alcohol | 8.00% |
| Denatured alcohol | 20.00% |
| Water | 64.35% |
| Glycerol | 2.00% |
| PEG-75 | 2.00% |
| PEG-60 hydrogenated castor oil | 0.60% |

Oil-in-Water Formulation Based on Essential Oil of ylang-Ylang:

| | |
|---|---|
| Essential oil of ylang-ylang | 0.2% |
| Sorbitol | 1.75% |
| Xanthan gum | 0.30% |
| Polyvinyl alcohol | 8.00% |
| Denatured alcohol | 20.00% |
| Water | 64.35% |
| Glycerol | 2.00% |
| PEG-75 | 2.00% |
| PEG-60 hydrogenated castor oil | 0.60% |

Other formulations may be prepared with an essential oil in an amount equal to 0.1% and 0.5% relative to the total weight of the composition.

The synthetic procedure for these three formulations entails:

preparing the aqueous phase by mixing together at 80-85° C. the water and the polyvinyl alcohol;

preparing the gelling phase by mixing together the xanthan gum and the glycerol until a uniform phase is obtained;

mixing together the above phases at a temperature of 80-85° C.;

lowering the temperature to 25-30° C. and successively incorporating, with stirring, the PEG-60 hydrogenated castor oil, the PEG-75, the essential oil and the denatured alcohol.

The varnish patch obtained is applied as a mask to the application point and is removed by peeling after a certain leave-on time, leaving the skin soft and cleansed.

### Example 3

### Preparation of a “Tonifying” Varnish Patch

Oil-in-Water Formulation Based on Essential Oil of Tea Tree:

| | |
|---|---|
| Essential oil of tea tree | 0.2% |
| Sorbitol | 1.75% |
| Xanthan gum | 0.30% |
| Polyvinyl alcohol | 8.00% |
| Denatured alcohol | 20.00% |
| Water | 64.35% |
| Glycerol | 2.00% |
| PEG-75 | 2.00% |
| PEG-60 hydrogenated castor oil | 0.60% |

Other formulations may be prepared with an essential oil in an amount equal to 0.1% and 5% relative to the total weight of the composition.

The procedure for this formulation entails:

preparing the aqueous phase by mixing together at 80-85° C. the water and the polyvinyl alcohol;

preparing the gelling phase by mixing together the xanthan gum and the glycerol until a uniform phase is obtained;

mixing together the above two phases at a temperature of 80-85° C.;

lowering the temperature to 25-30° C. and successively incorporating, with stirring, the PEG-60 hydrogenated castor oil, the PEG-75, the essential oil and the denatured alcohol.

The varnish patch obtained is applied as a mask to the application point and is removed by peeling after a certain leave-on time, leaving the skin soft and cleansed.

### Example 4

### Preparation of a “Relaxing” Hydrogel Patch

Hydrogel Formulation Based on Essential Oil of Lavender:

| | |
|---|---|
| Essential oil of lavender | 0.1-1% |
| Sodium polyacrylate | 5-10% |
| Ethyl para-hydroxybenzoate | 0.05-0.15% |
| Propyl para-hydroxybenzoate | 0.05-0.15% |
| Propylene glycol | 0.2-2% |
| Copolymer of starch and of sodium acrylate | 1-2% |
| Tartaric acid | 0.2-1% |
| Glycerol | 10-25% |
| Water | 60-75% |

Hydrogel Formulation Based on Essential Oil of Ylang-Ylang:

| | |
|---|---|
| Essential oil of ylang-ylang | 0.1-1% |
| Sodium polyacrylate | 5-10% |
| Ethyl para-hydroxybenzoate | 0.05-0.15% |
| Propyl para-hydroxybenzoate | 0.05-0.15% |
| Propylene glycol | 0.2-2% |
| Copolymer of starch and of sodium acrylate | 1-2% |
| Tartaric acid | 0.2-1% |
| Glycerol | 10-25% |
| Water | 60-75% |

The mode of preparation of the two formulations is as follows:

Compounds (3), (4) and (5) are mixed together and dissolved by heating, if necessary. Compound (8) is then added, followed by addition of compounds (1), (2) and (5). In another vessel, compound (7) is dissolved in (9) with stirring for 3 hours. The whole is then added to the above mixture. This mixture is then placed on a nonwoven support (polyester 65 g/m²) to form the patch.

Hydrogel Formulation Based on Essential Oil of Lavender:

| | |
|---|---|
| Essential oil of lavender | 0.5% |
| Sodium 2-acrylamido-2-methyl-propanesulfonate | 30% |
| Phenoxyethanol | 1% |
| Water | 68.5% |

Hydrogel Formulation Based on Essential Oil of Marjoram:

| | |
|---|---|
| Essential oil of marjoram | 0.5% |
| Sodium 2-acrylamido-2-methyl-propanesulfonate | 30% |
| Phenoxyethanol | 1% |
| Water | 68.5% |

These formulations are prepared as described above.

### Example 5

### Preparation of a “Tonifying” Hydrogel Patch

Hydrogel Formulation Based on Essential Oil of Tea Tree:

| | |
|---|---|
| Essential oil of tea tree | 0.1-1% |
| Sodium polyacrylate | 5-10% |
| Ethyl para-hydroxybenzoate | 0.05-0.15% |
| Propyl para-hydroxybenzoate | 0.05-0.15% |
| Propylene glycol | 0.2-2% |
| Copolymer of starch and of sodium acrylate | 1-2% |
| Tartaric acid | 0.2-1% |
| Glycerol | 10-25% |
| Water | 60-75% |

Hydrogel Formulation Based on Essential Oil of Mace:

| | |
|---|---|
| Essential oil of mace | 0.1-1% |
| Sodium polyacrylate | 5-10% |
| Ethyl para-hydroxybenzoate | 0.05-0.15% |
| Propyl para-hydroxybenzoate | 0.05-0.15% |
| Propylene glycol | 0.2-2% |
| Copolymer of starch and of sodium acrylate | 1-2% |
| Tartaric acid | 0.2-1% |
| Glycerol | 10-25% |
| Water | 60-75% |

The mode of preparation of the two formulations is as follows:

Compounds (3), (4) and (5) are mixed together and dissolved by heating, if necessary. Compound (8) is then added, followed by addition of compounds (1), (2) and (5). In another vessel, compound (7) is dissolved in (9) with stirring for 3 hours. The whole is then added to the above mixture. This mixture is then placed on a nonwoven support (polyester 65 g/m²) to form the patch.

The systems based on “relaxing” essential oil (lavender, marjoram or ylang-ylang) will be applied to the relaxing forehead point PC1, preferably in the evening before going to bed, onto skin that has been cleansed thoroughly.

The systems based on “tonifying” essential oil (tea tree or mace) will be applied to the tonifying cheekbone points 18-IG, preferably in the evening before going to bed, onto skin that has been cleansed thoroughly.

The application time is generally from 15 to 30 minutes, but it is possible to leave the compositions or patches to act overnight.

This application allows a diffusion of the essential oil into the epidermis at a specific acupuncture point, to modify the bioenergetic environment and to mimic the effect of acupuncture at the defined acupuncture point.

After a few weeks, the skin is visibly relaxed and smoothed out in the areas of tension and/or of formation of wrinkles (forehead) and firmed and tonified in the areas of slackening (cheekbones).

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for preventing and/or treating the signs of aging and/or stress of the skin, comprising topically applying onto at least one acupuncture point of the face or the neck of an individual in need of such treatment, at least one bioactive agent modifying the bioenergetic environment at said at least one acupuncture application point and thus mimicking the effects of acupuncture.

2. The regime or regimen as defined by claim 1, said at least one bioactive agent comprising a topically applicable cosmetic system including a physiologically acceptable medium therefor.

3. The regime or regimen as defined by claim 2, said at least one bioactive agent comprising a topically applicable cosmetic system for the transdermal delivery thereof.

4. The regime or regimen as defined by claim 2, said topically applicable cosmetic system having a surface adapted to the individual treatment of each of said acupuncture points.

5. The regime or regimen as defined by claim 2, said at least one acupuncture point of the face or the neck being selected from the group consisting of the points PC-1, 6-E, 18-IG, 19-GI, 23-VC, or a restricted area of the face or the neck comprising same.

6. The regime or regimen as defined by claim 2, said at least one bioactive agent comprising a trace element, a precious metal salt, a precious stone dust, a semi-precious stone dust, a vasoconstrictor, a vasodilator, an essential oil, or mixture thereof.

7. The regime or regimen as defined by claim 2, said topically applicable cosmetic system having a surface area ranging from 0.25 to 10 cm².

8. The regime or regimen as defined by claim 2, said topically applicable cosmetic system having a surface area ranging from 0.25 to 5 cm².

9. The regime or regimen as defined by claim 2, said topically applicable cosmetic system having a surface area ranging from 0.25 to 1 cm².

10. The regime or regimen as defined by claim 3, said topically applicable transdermal delivery system comprising at least one reservoir layer of bioactive agent permitting controlled release thereof through the skin.

11. The regime or regimen as defined by claim 2, said topically applicable cosmetic system comprising a patch, a hydrogel or a composite film.

12. The regime or regimen as defined by claim 2, said topically applicable cosmetic system comprising a self-adhesive solid or semi-solid.

13. The regime or regimen as defined by claim 1, said at least one bioactive agent modifying the bioenergetic environment of said at least one acupuncture application point being selected from the group consisting of bioactive agents acting on the electrophysiological status of the skin cells, agents modifying the electromagnetic environment of the skin cells, agents acting on the cutaneous capillary circulation, and mixtures thereof.

14. The regime or regimen as defined by claim 1, said at least one bioactive agent modifying the bioenergetic environment of said at least one acupuncture application point comprising at least one essential oil.

15. The regime or regimen as defined by claim 14, said at least one essential oil being selected from the group consisting of an essential oil eliciting a relaxing action, an essential oil of lavender, of ylang-ylang, of camomile, of sandalwood, of clary sage, of thyme, of marjoram, of grapefruit or of jasmine, an essential oil eliciting a tonifying action, an essential oil of eucalyptus, of vetiver, of lemon, of lemongrass, of litsea cubeba, of peppermint, of orange, of petitgrain, of ravensare, of bergamot, of tea tree or of mace, and a mixture thereof.

16. The regime or regimen as defined by claim 3, comprising topically applying onto the relaxing acupuncture points PC-1 and/or 6-E, or restricted area of the face comprising same, a transdermal delivery system which comprises a relaxing amount of at least one essential oil of lavender, of marjoram, of ylang-ylang, or mixture thereof.

17. The regime or regimen as defined by claim 3 for firming and/or tonifying the skin, comprising topically applying onto the tonifying acupuncture points 18-IG, 19-GI and/or 23-VC of the face and/or the neck, or a restricted area of the face and/or neck comprising same, a transdermal delivery system which comprises a tonifying amount of an essential oil of tea tree, of mace, or mixture thereof.

18. The regime or regimen as defined by claim 3 for simultaneously relaxing the forehead and firming the cheeks of the face, comprising topically applying onto the relaxing acupuncture point PC-1 or restricted area of the face comprising same, a transdermal delivery system which comprises a relaxing amount of at least one essential oil of lavender, of marjoram and/or of ylang-ylang, and also topically applying onto the tonifying acupuncture point 18-IG or a restricted area of the face comprising same, a transdermal delivery system which comprises a tonifying amount of at least one essential oil of tea tree and/or of mace.

19. The regime or regimen as defined by claim 3, said transdermal delivery system comprising from 0.01% to 20% by weight of at least one essential oil.

20. The regime or regimen as defined by claim 3, said transdermal delivery system further comprising a plant extract, a vitamin, or mixture thereof.

21. Cosmetic treatment kit comprising (1) at least one transdermal delivery system as defined by claim 3 containing at least one essential oil with a relaxing or tonifying action and (2) instructions for use indicating the location of the acupuncture points of the face and/or the neck, respectively, in (i) the areas of formation of wrinkles and/or of tension or (ii) the areas of slackening of the skin.
